(19) United States
(12) Patent Application Publication
(10) Pub. No.: US 2023/0415070 A1
(43) Pub. Date: Dec. 28, 2023

(54) EUTECTIC COMPOSITIONS, METHODS AND USES THEREOF

(71) Applicant: Universidade do Minho, Braga (PT)

(72) Inventors: Artur Manuel CAVACO PAULO, Braga (PT); Carla Manuela PEREIRA MARINHO DA SILVA, Guimãres (PT); David DA SILVA FREITAS, Braga (PT); Jennifer NORO, Viana do Castelo (PT)

(21) Appl. No.: 18/252,712

(22) PCT Filed: Nov. 15, 2021
(86) PCT No.: PCT/EP2021/081751

§ 371 Date: May 11, 2023

(30) Foreign Application Priority Data
Nov. 13, 2020 (PT) 116885

(51) Int. Cl. B01D 11/02; A61K 8/64; A61Q 19/00; A61Q 5/00

(52) U.S. Cl. CPC B01D 11/0288; A61K 8/64; A61K 2800/80; A61Q 5/00; A61Q 19/00

(57) ABSTRACT

The present disclosure relates to a natural deep eutectic mixture for extraction of biocomponents comprising two different solvents, wherein a first solvent is selected from a list consisting of: lactic acid, and glycerol, citric acid, maleic acid, and tetrabutylammonium bromide and a second solvent is selected from a list consisting of: sodium lactate, sodium citrate, transcutol, glycine, glycerol, oleic acid, sodium lactate, decanoic acid; further comprising up to 90% (w/w) in water. The disclosure also relates to a method to obtain an extract from a natural source material using the NADES, as well as compositions comprising the NADES, the obtained extract and/or a topical active compound. The use of said compositions as cosmetic formulations is also described.

### TECHNICAL FIELD

The present disclosure relates to the development of Natural Deep Eutectic Solvents (NADES) using natural products, like sugars, organic bases and organic acids, as starting compounds. These solvents can be used for the extraction of bioactive compounds from natural sources, such as cork; agricultural wastes, including grape seed and peels; tomato; olive oil; and plants (teas, eucalyptus, lavender, or others), and from fish skin and bones. The extractives could then be further formulated with active topical cosmetic components to prepare cosmetic compositions.

### BACKGROUND

Environmental issues have driven the search for green safer solvents to replace harsh solvents on chemical processes. Sugars, amino acids or organic acids are typically solid at room temperature. When combined in a specific molar ratio, they have a high melting point depression, thus becoming liquid at room temperature. These particular mixtures are called Natural Deep Eutectic Solvents—NADES.

NADES have been reported for the first time by Choi et al. in 2011 as an alternative to Ionic Liquids (IL) and Deep Eutectic Solvents (DES) [1]. Since then, several NADES, composed by sugars, amino acids and organic acids have been described in literature [1-4]. NADES have the ability to dissolve natural or synthetic chemicals with low solubility in water, and their properties such as polarity, viscosity, biodegradability, electrical conductivity and thermal stability, can be altered by changing one of its components and molar ratio, as well as by addition of a co-solvent [5]. NADES applications go beyond chemical or materials engineering and cover a wide range of fields from biocatalysis, extraction, electrochemistry, carbon dioxide, synthesis, degradation, dyeing or biomedical applications [6-8].

Natural extracts reveal the potential of natural sources to provide a plentiful of chemical compounds which, due to synergistic effects, have a much more powerful activity when compared to the single molecules. A broad range of bioactive chemical compounds can be derived from plants, either in their pure form or as homogenous extracts. As these compounds have broad structural and functional diversities, they offer cosmetic and pharmaceutical opportunities for the development of new products. They may also represent an excellent source of molecules for the production of food additives, functional foods, nutritional products, and nutraceuticals.

Since NADES can dissolve both polar and non-polar metabolites they can serve as solvents for the extraction of many types of natural compounds depending on their physicochemical properties [2]. An extraction process using NADES was suggested by Dai et al., who studied the extraction of phenolics from safflower [9-10]. NADES can also be used for the extraction and dissolution of some lignocellulosic materials [11]. The use of NADES in the extraction processes from natural sources can lead to highly efficient and truly ecological extraction methods. The stability of phenolic compounds in NADES and the biological activity of the extracts will have to be studied for a better application of these solvents in the extraction of natural compounds [12]. In some cases, described in literature, it was shown that the extraction yields may double the obtained with organic solvents [9-11]. Researchers have established that the extraction process is affected by several factors, such as the molar ratio between the initial NADES molecules, the affinity between the DES and the target compounds, the water content and the extraction conditions [2-3].

The separation of the extractives from NADES represents a great challenge due to the strong hydrogen bond network established between them [12]. It has been reported in literature several methods for the separation involving the use of liquid/liquid extractions and distillations by drag steam, using solvents such as water, ethanol, ethyl acetate, among others [13]. Examples of NADES applications include the extraction of bioactive compounds from natural biopolymers like lignocellulosic biomass, starch, cellulose [7], wool keratin [14], agricultural wastes among others. The extracts containing NADES can be applied directly in cosmetic and pharmaceutical formulations, if they are stable and compatible at the biological level [16]. Another possible direct application of the extracts is in polymerization processes for the production of new biomaterials [17].

The extractives composition could be further formulated by adding on active components for cosmetic topical applications.

These facts are disclosed in order to illustrate the technical problem addressed by the present disclosure.

### GENERAL DESCRIPTION

The present disclosure is related to the development of new natural deep eutectic solvents (NADES), or natural deep eutectic mixtures, using natural components, and their application in the extraction of bioactive compounds from several natural sources.

The new NADES are made-up with two natural components mixed in different ratios using temperature to help dissolution. The components of the natural deep eutectic mixture include the ethylene glycol, lactic acid, glycerol, sodium citrate, sodium lactate, caprylic acid, and enanthic acid. The mixtures developed have a lower melting point than the isolated compounds. The constituents of the disclosed NADES are non-toxic and compatible with the living tissues, being therefore suitable for applications in the cosmetic or pharmaceutical fields.

In an embodiment, the present disclosure is focused on the application of NADES for the extraction of chemical compounds from natural sources. The extraction methods applied are the “enfleurage” method, ultrasound-assisted extraction and the sealed system extraction. The natural extracts obtained can be applied directly in cosmetic formulations without further purification.

The present disclosure relates to a natural deep eutectic mixture for extraction of biocomponents comprising two different solvents, wherein a first solvent is selected from a list consisting of: lactic acid, ethylene glycol, glycerol, caprylic acid, enanthic acid, glucose, transcutol, citric acid, menthol, sodium lactate, sodium acetate, xylitol, sorbitol, decanoic acid, maleic acid, malic acid, oxalic acid, tartaric acid, oleic acid, palmitic acid and tetrabutylammonium bromide; and a second solvent is selected from a list consisting of: ethylene glycol, sodium lactate, sodium citrate, caprylic acid, enanthic acid, transcutol, glycine, glycerol, glucose, oleic acid, formic acid, sodium acetate and decanoic acid. Better results are obtained when the mixture is not lactic acid as a first solvent and glucose as a second solvent.

Another aspect of the present disclosure relates to a natural deep eutectic mixture for extraction of biocomponents comprising two different solvents, wherein a first solvent is selected from a list consisting of: lactic acid, and glycerol, citric acid, maleic acid, and tetrabutylammonium bromide; and a second solvent is selected from a list consisting of: sodium lactate, sodium citrate, transcutol, glycine, glycerol, oleic acid, sodium lactate, decanoic acid; further comprising up to 90% (w/w) in water. The disclosure also relates to a method to obtain an extract from a natural source material using the NADES, as well as compositions comprising the NADES, the obtained extract and/or a topical active compound. The use of said compositions as cosmetic formulations is also described.

In an embodiment, the natural deep eutectic mixture comprises:
20-90% (mol/mol) of the first solvent; preferably 30-80% (mol/mol);
10-80% (mol/mol) of the second solvent, preferably 12-50% (mol/mol).

In an embodiment, the natural deep eutectic mixture is selected from the following list:
lactic acid and glycerol; lactic acid and sodium citrate; glycerol and sodium lactate;
glycerol and transcutol; lactic acid and transcutol; transcutol and sodium lactate;
lactic acid and glycine; tetrabutylammonium bromide and oleic acid;
tetrabutylammonium bromide and decanoic acid; preferably the natural deep eutectic mixture is a combination of: glycerol and transcutol, or lactic acid and transcutol, glycerol and maleic acid, or mixtures thereof.

In an embodiment, the natural deep eutectic mixture is a combination of: lactic acid and ethylene glycol, or lactic acid and glycerol, or ethylene glycol and sodium lactate, or glycerol and sodium lactate, or caprylic acid and ethylene glycol, or lactic acid and caprylic acid, or enanthic acid and ethylene glycol, or enanthic acid and glycerol, or lactic acid and enanthic acid, or glucose and ethylene glycol, or glucose and glycerol, or glucose and sodium lactate, or ethylene glycol and transcutol, or glycerol and transcutol, or lactic acid and transcutol, or transcutol and sodium lactate, or menthol and transcutol, or glycerol and formic acid, or ethylene glycol and formic acid, or lactic acid and formic acid, sodium lactate and formic acid, transcutol and formic acid, sodium acetate and formic acid, xylitol and formic acid, xylitol and sodium acetate, sorbitol and formic acid, sorbitol and sodium acetate, ethylene glycol and sodium acetate, transcutol and sodium acetate, lactic acid and sodium acetate, sodium lactate and sodium acetate, citric acid and glycerol, citric acid and ethylene glycol, citric acid and transcutol, caprylic acid and transcutol, decanoic acid and transcutol, enanthic acid and transcutol, oleic acid and transcutol, decanoic acid and ethylene glycol, maleic acid and ethylene glycol, malic acid and ethylene glycol, malic acid and glycerol, oxalic acid and glycerol, oxalic acid and ethylene glycol, tartaric acid and glycerol, tartaric acid and ethylene glycol, in an equivalent molar ratio of 1:1.

In another embodiment, the natural deep eutectic mixture comprises lactic acid and glycerol in an equivalent molar ratio of 1:4 to 4:1, preferably (1:4 to 1:3); more preferably 1:1.

In another embodiment, the natural deep eutectic mixture comprises lactic acid and glycerol in an equivalent molar ratio of 1:4 to 4:1, preferably 4:1.

In yet another embodiment, the natural deep eutectic mixture comprises lactic acid and sodium citrate in an equivalent molar ratio of 2:1 to 8:1, preferably 2:1.

In another embodiment, the natural deep eutectic mixture comprises ethylene glycol and sodium lactate, or glycerol and sodium lactate, in an equivalent molar ratio of 1:2 to 2:1, preferably 1:2.

In another embodiment, the natural deep eutectic mixture comprises citric acid and sodium lactate in an equivalent molar ratio of 1:4 to 1:3.

In another embodiment, the natural deep eutectic mixture comprises lactic acid and glycine in an equivalent molar ratio of 5:1.

In an embodiment, the natural deep eutectic mixture comprises tetrabutylammonium bromide and oleic acid, or tetrabutylammonium bromide and decanoic acid, or maleic acid and ethylene glycol, in an equivalent molar ratio of 1:2.

In an embodiment, the natural deep eutectic mixture comprises caprylic acid and ethylene glycol, or lactic acid and caprylic acid, or decanoic acid and ethylene glycol, in an equivalent molar ratio of 2:1.

In an embodiment, the natural deep eutectic mixture further comprises up to 90% (w/w) in water, preferably 6 to 20% (w/w) in water.

In another embodiment, the natural deep eutectic mixture is clear and liquid at a temperature ranging from 15 to 30° C.

In an embodiment, the melting point of the natural deep eutectic mixture ranges from −55 to −15° C., preferably from −52 to −20° C.

In an embodiment, the pH of the natural deep eutectic mixture varies from 1 to 10, preferably ranges from 2 to 7.

In an embodiment, the pH is adjustable by changing the molar ratio between the two different solvents. In another embodiment, the pH is adjustable by the addition of sodium hydroxide, preferably 10 to 30% (w/w) (weight of sodium hydroxide/weight of mixture).

In an embodiment, the density of the natural deep eutectic mixture ranges from 1.2 to 1.4 g·ml⁻¹.

In an embodiment, the conductivity of the natural deep eutectic mixture ranges from 0.002 to 1.6 mS·cm⁻¹, preferably from 0.002 to 0.8 mS·cm⁻¹.

In an embodiment, the viscosity at 25° C. of the natural deep eutectic mixture increases by the creation of ester or amide bonds between the first and the second solvent. These bonds can be formed by reacting the natural deep eutectic mixtures with a lipase, esterase or protease. After the reaction, the enzymes are removed from the mixture.

In the state of the art, the viscosity may be measured by many methods. In the present disclosure the viscosity measurement of the eutectic composition was carried out in a Brookfield DV-II+Pro equipment using a 500 mL glass beaker containing the composition of the present disclosure up to its maximum capacity, the viscosity measurement being carried out with SC4-27 or SC4-28 spindles, a rotation of 50 rpm, and a torque between 10% and 100%, in particular 10% and 50%, at 25° C.

In an embodiment, the viscosity of the deep eutectic mixture ranges from 0.015 to 1700 Pa·s at 25° C.

In an embodiment, the refractive index of the natural deep eutectic mixture ranges from 1.4 to 1.5.

In an embodiment, the natural deep eutectic mixture further comprises a topical active compound. In a further embodiment, the topical active compound is selected from a list comprising: icilin, menthol, carboxylated icilin, carboxylated menthol, 2,6-dimethylaniline, Carboxyiciline-2,6 dimethylaniline conjugate, Carboxymenthol-2,6-dimethylaniline conjugate, Dermorphin-derived tetrapeptide (Dmt1) DALDA, Carboxymenthol-DALDA, Carboxyicilin-DALDA, opioid peptides, Carboxymenthol-YGGFL conjugate, Carboxymenthol-YGGFM conjugate, Carboxymenthol-YPWF-NH2 conjugate, Carboxymenthol-YPFF-NH2 conjugate, Carboxyicilin-YGGFL conjugate, Carboxyicilin-YGGFM conjugate, Carboxyicilin-YPWF-NH2 conjugate, Carbocyicilin-YPFF-NH2 conjugate, Sivelestat, Argireline Ac-EEMQRR-NH2, Sivelestate-argireline Ac-EEMQRR-NH2, secretory leukocyte protease inhibitor, or mixtures thereof. A topical active compound is a compound that may be used in used in topical treatment, namely on skin/hair.

The present disclosure also relates to a method to obtain an extract from a natural source material, comprising the following steps: contacting the natural source material with a natural deep eutectic mixture described in any of the previous claims, preferably by dipping; incubating the natural deep eutectic mixture and the natural source material at a temperature ranging from 25° C. to 150° C., preferably 25 to 100° C.; replacing the used natural source material by a new natural source material; repeating the previous steps for 0 to 30 times.

In an embodiment, the step of incubating the natural deep eutectic mixture and the natural source material may be at a range temperature from 25° C. to 80° C.

In an embodiment, the step of incubating the natural deep eutectic mixture and the natural source material may occur during 1 min-30 days. Preferably for 1 day-10 days.

In an embodiment, the step of incubating the natural deep eutectic mixture and the natural source material may be at room temperature for 1-30 days, preferably by the enfleurage method.

In an embodiment, the step of incubating the natural deep eutectic mixture and the natural source material may be at 25° C. to 80° C. for 1 minute to 24 hours, preferably in an ultrasonic bath.

In embodiment, the step of incubating the natural deep eutectic mixture and the natural source material may be at 25° C. to 150° C. for 1 minute to 24 hours, preferably in a sealed system.

In an embodiment, the natural source material is selected from a list comprising cork, agricultural wastes (including tomato, olive oil, grape seeds, grape peels), plants (such as teas, eucalyptus, lavender), fish skin and bones, or mixtures thereof. In a further embodiment, the natural source material is cork.

An aspect of the present disclosure relates to an extract obtainable by the method described in the present document. In an embodiment, the extract is in solution, in suspension or lyophilized.

In an embodiment, the extract comprises fatty acids and oils, such as oleic acid, palmitic acid, stearic acid, 1-docosanol; alcohols and small acids, such as 2-hydroxymalonic acid, 2,3-Butanediol; phenolics and aromatics, such as ferulic acid and derivatives, diisooctyl phthalate or analogues; terpenoids, such as borneol or pantolactone; sugars, such as D-sorbitol, D-mannonic acid; steroids, such as friedelin, stigmasterol; or mixtures thereof.

In an aspect, the present disclosure also relates to a composition comprising the natural deep eutectic mixture and at least one of the following: the extract obtainable by the method described in the present document, or up to 1% (w/w) of topical active compound. In an embodiment, the composition comprises up to 90% (w/w) of the natural deep eutectic mixture, up to 10% (w/w) of the extract and up to 1% (w/w) of the topical active compound.

In an embodiment, the natural deep eutectic mixture may further comprise 0.01-1% (w/w) of the topical active compound; preferably 0.1-0.5 (w/w) of the topical active compound.

In an embodiment, the topical active compound is selected from a list comprising: icilin, menthol, carboxylated icilin, carboxylated menthol, 2,6-dimethylaniline, Carboxyiciline-2,6 dimethylaniline conjugate, Carboxymenthol-2,6-dimethylaniline conjugate, Dermorphin-derived tetrapeptide (Dmt1) DALDA, Carboxymenthol-DALDA, Carboxyicilin-DALDA, opioid peptides, Carboxymenthol-YGGFL conjugate, Carboxymenthol-YGGFM conjugate, Carboxymenthol-YPWF-NH2 conjugate, Carboxymenthol-YPFF-NH2 conjugate, Carboxyicilin-YGGFL conjugate, Carboxyicilin-YGGFM conjugate, Carboxyicilin-YPWF-NH2 conjugate, Carbocyicilin-YPFF-NH2 conjugate, Sivelestat, Argireline Ac-EEMQRR-NH2, Sivelestate-argireline Ac-EEMQRR-NH2, secretory leukocyte protease inhibitor, or mixtures thereof (compounds listed in Table 5).

In an embodiment, the composition may further comprise a component selected from a list consisting of: hyaluronic acid, niacinamide, folic acid, D-panthenol, tocopherol, ceramide NP (3), ceramide AP (6 II), ceramide EOP (1), apigenin, quercitin, luteolin, ursolic acid, rosmarinic acid, thymol, carvacrol, cooper peptide, K18 peptide, retinol, urea, xylitol, or mixtures thereof.

In an embodiment, the opioid peptide may be selected from the following list:

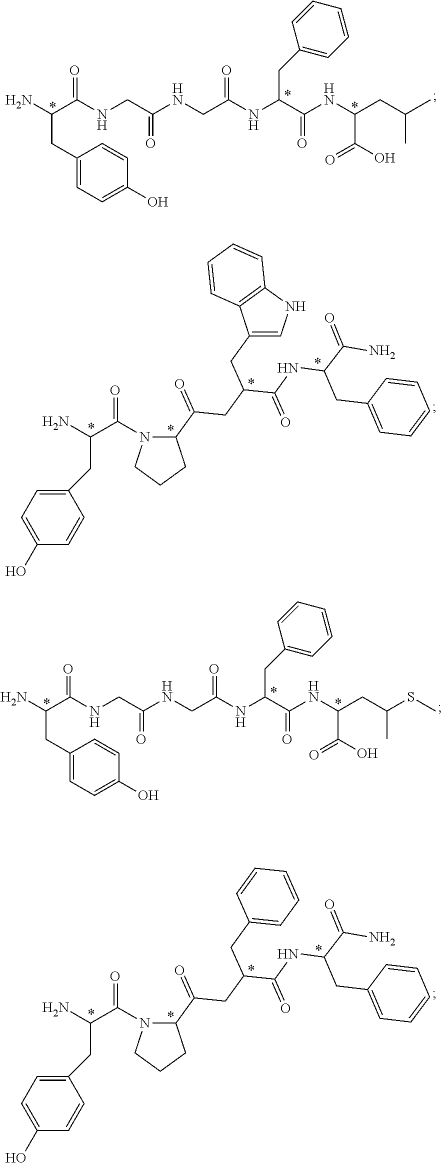 [SMILES: CC(C)CC(NC(=O)C(Cc1ccccc1)NC(=O)CNC(=O)CNC(=O)C(N)Cc1ccc(O)cc1)C(=O)O ; NC(=O)C(Cc1ccccc1)NC(=O)C(CC(=O)C1CCCN1C(=O)C(N)Cc1ccc(O)cc1)Cc1c[nH]c2ccccc12 ; CSC(C)CC(NC(=O)C(Cc1ccccc1)NC(=O)CNC(=O)CNC(=O)C(N)Cc1ccc(O)cc1)C(=O)O ; NC(=O)C(Cc1ccccc1)NC(=O)C(CC(=O)C1CCCN1C(=O)C(N)Cc1ccc(O)cc1)Cc1ccccc1]

or mixtures thereof.

The present disclosure also relates to the use of the composition as described as a cosmetic formulation.

In an embodiment, the composition may be used in hair treatment, namely hair products, preferably in hair conditioners, hair curling agents, hair straightening agents, hair masks, or hair shampoos.

In an embodiment, the composition may be used in skin care treatment, namely skin care products, preferably in skin balms, skin creams, skin soap, skin masks, or skin moisturizers.

### BRIEF DESCRIPTION OF THE DRAWINGS

The following figures provide preferred embodiments for illustrating the disclosure and should not be seen as limiting the scope of invention.

FIG. 1: Representation of an embodiment of the coloration of the extracts obtained after extraction using NADES as extraction media. A—Formulation NADES 2 before the extraction process; B—Enfleurage method extraction using the formulation NADES 2; C—Ultrasound assisted extraction using the formulation NADES 2; D—Sealed system extraction using the formulation NADES 2.

FIG. 2: Representation of an embodiment of the extraction of cork using NADES as extraction media; at the left side is represented the raw material and at the right side the liquid and the remaining material after extraction.

FIG. 3: Representation of an embodiment of the extraction of tomato using NADES as extraction media; at the left side is represented the raw material and at the right side the liquid and the remaining material after extraction.

FIG. 4: Representation of an embodiment of the extraction of fish skin using NADES as extraction media; at the left side is represented the raw material and at the right side the liquid and the remaining material after extraction.

FIG. 5: Representation of an embodiment of the extraction of grape bunch using NADES as extraction media; at the left side is represented the raw material and at the right side the liquid and the remaining material after extraction.

### DETAILED DESCRIPTION

The present disclosure relates to natural deep eutectic mixtures (NADES) comprising two different solvents, wherein a first solvent is selected from a list consisting of: lactic acid, ethylene glycol, glycerol, caprylic acid, enanthic acid, glucose, transcutol, citric acid, menthol, sodium lactate, sodium acetate, xylitol, sorbitol, decanoic acid, maleic acid, malic acid, oxalic acid, tartaric acid, oleic acid, palmitic acid, and tetrabutylammonium bromide; and a second solvent is selected from a list consisting of: ethylene glycol, sodium lactate, sodium citrate, caprylic acid, enanthic acid, transcutol, glycine, glycerol, glucose, oleic acid, formic acid, sodium acetate and decanoic acid. The disclosure also relates to a method to obtain an extract from a natural source material using the NADES, as well as compositions comprising the NADES, the obtained extract and/or a topical active compound. The use of said compositions as cosmetic formulations is also described.

The present disclosure relates to the use of natural deep eutectic solvents (NADES) in the extraction of bioactive compounds from Quercus suber (cork), agricultural wastes like grape peels and seeds, tomato, olive oil, and plants (teas, eucalyptus, lavender) and fish skin and bones. Extracts of natural products are often used in the cosmetic and pharmaceutical industry. In an embodiment, all the components used to prepare the NADES are approved to be incorporated in cosmetic formulations. The extracts obtained can be use directly in cosmetic formulations without any purification step.

In an embodiment, the biomaterial chosen for the application of NADES as extraction solvents was Quercus suber cork (Cork oak). This natural material is constituted by suberin (=42%), lignin (=22%), polysaccharides (=20%), some extractives compounds (=15%) and ash (=1%). Suberin is a complex lipophilic biopolymer mainly composed of long chain fatty acids called suberin acids, some alcohols like glycerol and polyaromatic compounds [18-20]. All these components can be found in cosmetic and/or pharmaceutical formulations, which is why the scientific community is interested in extracting this type of natural compounds from cork.

In an embodiment, new NADES were formed by the mixing of at least two compatible natural compounds at proper ratios (Table 1). These compounds strongly interact by hydrogen bonds interactions to form the liquid. The combination of all the constituents results in a high melting point depression that gives the new NADES. The formation temperatures of these solvents do not exceed 100° C.

TABLE 1
NADES composition.

| NADES | | | Ratio |
|---|---|---|---|
| Composition | Eutectic mixture | | (molar ratio) |
| 1 | Lactic Acid | Ethylene glycol | 1:1 |
| 2 | Lactic Acid | Glycerol | 4:1; 2:1; 1:1; |
| | | | 1:2; 1:4 |
| 3 | Lactic Acid | Sodium Citrate | 2:1; 4:1; |
| | | (monobasic to | 6:1; 8:1 |
| | | tribasic) | |
| 4 | Ethylene glycol | Sodium Lactate | 2:1; 1:1; 1:2 |
| 5 | Glycerol | Sodium Lactate | 2:1; 1:1; 1:2 |
| 6 | Caprylic acid | Ethylene glycol | 1:1; 2:1 |
| 7 | Lactic Acid | Caprylic acid | 1:1; 2:1 |
| 8 | Enanthic acid | Ethylene glycol | 1:1 |
| 9 | Enanthic acid | Glycerol | 1:1 |
| 10 | Lactic Acid | Enanthic acid | 1:1 |
| 11 | Glucose | Ethylene glycol | 1:1 |
| 12 | Glucose | Glycerol | 1:1 |
| 13 | Glucose | Sodium Lactate | 1:1 |
| 14 | Ethylene glycol | Transcutol | 1:1 |
| 15 | Glycerol | Transcutol | 1:1 |
| 16 | Lactic Acid | Transcutol | 1:1 |
| 17 | Transcutol | Sodium Lactate | 1:1 |
| 18 | Citric acid | Sodium Lactate | 1:3; 1:4 |
| 19 | Lactic acid | Glycine | 5:1 |
| 20 | Tetrabutylammonium | Oleic acid | 1:2 |
| | bromide | | |
| 21 | Tetrabutylammonium | Decanoic acid | 1:2 |
| | bromide | | |
| 22 | Menthol | Transcutol | 1:1 |
| 23 | Glycerol | Formic Acid | 1:1 |
| 24 | Ethylene glycol | Formic Acid | 1:1 |
| 25 | Lactic Acid | Formic Acid | 1:1 |
| 26 | Sodium Lactate | Formic Acid | 1:1 |
| 27 | Transcutol | Formic Acid | 1:1 |
| 28 | Sodium Acetate | Formic Acid | 1:1 |
| 29 | Xylitol | Formic Acid | 1:1 |
| 30 | Xylitol | Sodium Acetate | 1:1 |
| 32 | Sorbitol | Formic Acid | 1:1 |
| 33 | Sorbitol | Sodium Acetate | 1:1 |
| 33 | Ethylene glycol | Sodium Acetate | 1:1 |
| 34 | Transcutol | Sodium Acetate | 1:1 |
| 35 | Lactic Acid | Sodium Acetate | 1:1 |
| 36 | Sodium Lactate | Sodium Acetate | 1:1 |
| 37 | Citric Acid | Glycerol | 1:1; 1:2; 1:3 |
| 38 | Citric Acid | Ethylene glycol | 1:1; 1:2; 1:3 |
| 39 | Citric Acid | Transcutol | 1:1; 1:2; 1:3 |
| 40 | Caprylic Acid | Transcutol | 1:1 |
| 41 | Decanoic Acid | Transcutol | 1:1 |
| 42 | Enanthic Acid | Transcutol | 1:1 |
| 43 | Oleic Acid | Transcutol | 1:1 |
| 44 | Decanoic Acid | Ethylene glycol | 1:1; 2:1 |
| 45 | Maleic Acid | Ethylene glycol | 1:1; 1:2 |
| 46 | Malic Acid | Ethylene glycol | 1:1 |
| 47 | Malic Acid | Glycerol | 1:1 |
| 48 | Oxalic Acid | Glycerol | 1:1 |
| 49 | Oxalic Acid | Ethylene glycol | 1:1 |
| 50 | Tartaric Acid | Glycerol | 1:1 |
| 51 | Tartaric Acid | Ethylene glycol | 1:1 |

In an embodiment, the NADES presented in table 1 were prepared by mixing the constituents at temperatures ranging from 25° C. to 100° C., under vigorous stirring. After 1 hour, a clear solution was formed, and the eutectic mixture was kept at room temperature for further use. Depending on the application, it is possible to add some amount of water (0-90% (w/w)) to modulate the properties of the eutectic mixtures.

For the scope and interpretation of the present disclosure it is defined that “room temperature” should be regarded as a temperature between 15-30° C., preferably between 18-25° C., more preferably between 20-22° C.

In an embodiment, the components of the eutectic mixture include ethylene glycol, lactic acid, glycerol, sodium citrate, sodium lactate, caprylic acid, enanthic acid, glucose and transcutol, among others. All of these components are non-toxic and biocompatible, being therefore suitable for the future applications in the cosmetic or pharmaceutical fields.

Some physical-chemical properties of the eutectic mixtures where measured, such as the melting point, pH, density, conductivity and refractive index (table 2). These new NADES have a lower melting point than the isolated constituents; the pH can be modulated by changing the ratio between NADES components; and the NADES physicochemical properties can also be slightly modulated by changing the ratio between the components.

TABLE 2
Physical/chemical properties of the NADES used in examples.

| | Eutectic | | Melting | | Density | Conductivity | Refractive |
|---|---|---|---|---|---|---|---|
| NADES | mixture | Ratio | point (° C.) | pH | (g · mL⁻¹) | (mS · cm⁻¹) | index |
| 2 | Glycerol:Lactic | 1:1 | −52 | 1 | 1.2424 | 0.00295 | 1.45727 |
| | acid | | | (6.6% | | | |
| | | | | H₂O) | | | |
| 3 | Sodium | 4:1 | −15 | 5 | 1.3920 | 1.58 | 1.43809 |
| | citrate tribasic | | | (12.6% | | (30% | |
| | dihydrate:Lactic | | | H₂O) | | H₂O) | |
| | acid | | | | | | |
| 4 | Ethylene | 1:1 | −19 | 7.6 | 1.3433 | 0.722 | 1.45507 |
| | glycol:Sodium | | | (20% | | | |
| | lactate | | | H₂O) | | | |
| 5 | Glycerol:Sodium | 1:1 | −21 | 7.9 | 1.3658 | 0.0487 | 1.46670 |
| | lactate | | | (20% | | | |
| | | | | H₂O) | | | |

In an embodiment, the mixtures developed have a lower melting point than the isolated constituents. All NADES presented in this work are liquid at room temperature. With the addition of sodium hydroxide (NaOH), it was possible to increase the pH of NADES and these remained liquid at room temperature. As an example, the addition of granules of 24% NaOH (mass of NaOH/mass of NADES), the pH of NADES 2 increases from 1 to 7 without changing its physical state.

An aspect of the present disclosure focuses on the application of NADES in the extraction of chemical compounds from natural sources. NADES were used as solvents for the extraction of these compounds following the same methodology principle designated as “enfleurage” replacing the fat by NADES [21]. Other extraction techniques, like Ultrasound assisted extraction and sealed system extraction, were also used.

In an embodiment, the NADES were used to extract chemical compounds from cork, agricultural wastes and plants, preferably to extract alcohols, fatty acids, phenolics, steroids, terpenoids, or sugars.

In an embodiment, the enfleurage method was performed using cork from Quercus suber (0.1 g-1 Kg). The cork was placed in a recipient and submerged with the eutectic solvent (0.1 mL to 25 L) and with/without a water percentage (0-90% (w/w)). The system was covered and left at room temperature for 1-30 days. After this process, the old cork was removed and a new one was added. The same process was repeated for 0 to 30 times.

In another embodiment, the extraction was performed using the ultrasonic bath assisted extraction. Cork from Quercus suber (0.1 g-1 Kg) was placed in a recipient, and the eutectic solvent (0.1 mL-25 L) was added with or without a water percentage (0-90% (w/w)). The system was covered and put in the ultrasonic bath for 1 minute to 24 hours and at temperatures ranging from 25° C. to 80° C. After this process, the old cork was removed and a new one was added. The same process was repeated for 0 to 30 times.

In a yet further embodiment, the extraction was performed using the sealed system extraction. Cork from Quercus suber (0.1 g-1 Kg) was placed in amber flask and was added 5 mL of the NADES extraction solvent (0.1 mL-25 L) and with/without a water percentage (0-90% (w/w)). The flask was sealed with an aluminium seal cap and put in an oil bath, under magnetic agitation, at temperatures ranging from 25° C. to 150° C. for 1 minute to 24 hours. After this process, the old cork was removed and a new one was added. The same process was repeated for 0 to 30 times.

In an embodiment, at the end of each extraction process, all remaining cork was washed with water to extract the solvent that may be adsorbed by the biomaterial by a natural extract treatment. The remaining water content in the extract was removed by evaporation under pressure. The extract containing the eutectic mixture was obtained for further application.

The extraction yields increased when NADES were used as solvents instead of water. From the applied methods, the sealed system is the most efficient extraction method, followed by extraction assisted by ultrasound and, finally, the enfleurage method showed lower extraction efficiency. In an embodiment, when NADES 2 was used as a solvent, the extraction yields were 12% for sealed system, 6.9% for ultrasound assisted method, and 3.3% for the enfleurage method. Additionally, when NADES 2 was used as solvent in the enfleurage method, the extraction yield was 4 times higher when compared to water under the same conditions. For the ultrasound-assisted method, the yield was 2 times higher and, for the sealed system, it was 3 times. Table 3 lists a comparison of extraction yields obtained in three different extraction methods when using water or NADES 2 as solvent.

TABLE 3
Extraction yields for the three extraction methods using
formulation NADES 2 or water as solvent; the yield of extraction
was obtained by the difference between the total initial
cork mass and the mass obtained after extraction.

| | | | | Ultrasound | Sealed |
|---|---|---|---|---|---|
| | Eutectic | Eutectic | Enfleurage* | assisted** | system*** |
| Solvent | compounds | Ratio | Yield | Yield | Yield |
| H₂O | — | — | 0.7% | 3.2% | 4.2% |
| NADES | Lactic acid: | 1:1 | 3.3% | 6.9% | 18.6% |
| 2 | Glycerol | | | | |
| NADES | Ethylene | 1:1 | 3.5% | 8.5% | 23.0% |
| 4 | glycol: | | | | |
| | Sodium | | | | |
| | Lactate | | | | |
| NADES | Glycerol: | 1:1 | 3.9% | 9.0% | 25.8% |
| 5 | Sodium | | | | |
| | Lactate | | | | |

*Enfleurage method: Cork 0.7 g; Solvent 15 mL; 0% of water in NADES; room temperature; 3 days; 3 cycles.
**Ultrasound assisted: Cork 0.7 g; Solvent 10 mL; 0% of water in NADES; 50° C.; 6 hours; 3 cycles.
***Sealed system: Cork 0.7 g; Solvent 5 mL; 0% of water in NADES; 100° C. 6 hours; 3 cycles.

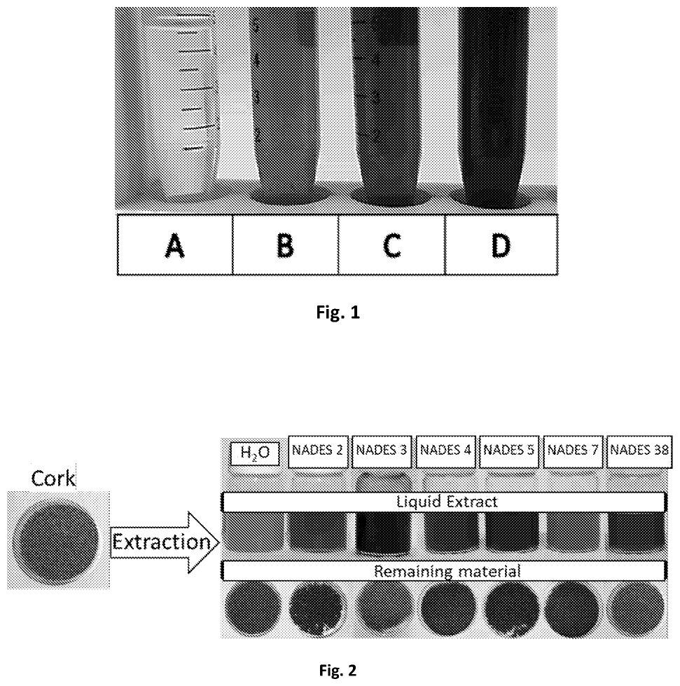

FIG. 1 shows a representation of an embodiment of the coloration of the extracts obtained after extraction using NADES as extraction media. Formulation of NADES 2 (FIG. 1-A) were used to perform the extraction process, using different methods. FIG. 1-B shows the extract obtained from the enfleurage method extraction using the formulation NADES 2, using 0.7 g of cork; 15 mL of the natural deep eutectic mixture without the addition of water in the mixture. The extraction was performed at room temperature, for 3 days in 3 cycles. FIG. 1-C shows an embodiment of an ultrasound assisted extraction using the formulation NADES 2, 0.7 g of cork; 10 mL of the natural deep eutectic mixture, without the addition of water in the mixture, at 50° C.; for 6 hours in 3 cycles. FIG. 1-D shows the extract obtained after sealed system extraction using the formulation NADES 2, 0.7 g of cork, 5 mL of the natural deep eutectic mixture, without the addition of water in the mixture, at 100° C. for 6 hours, in 3 cycles.

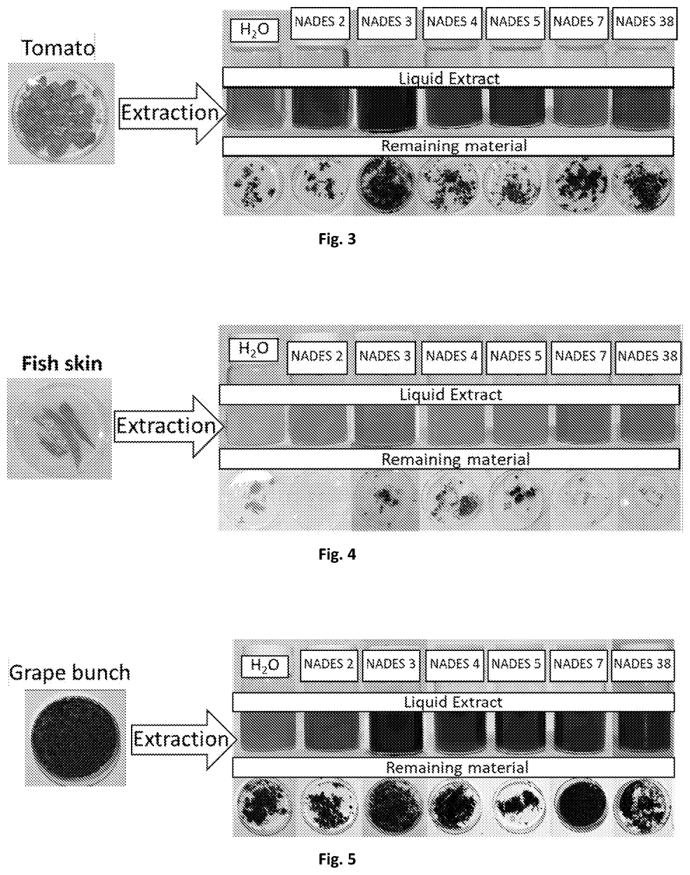

In an embodiment, the colour increment (FIG. 1) of the extracts obtained from cork using NADES is related to the increase of the extract concentration. The sealed system method provides the most effective extraction of natural cork compounds. As an example, for the extraction using the sealed system at 100° C., 5 mL of NADES 2 were used to extract from 2.1 g of cork (3 cycles of 0.700 g of renewal of cork). From this extraction, the final extract concentration obtained was 0.07812 g/mL (m_{extract}/vₛₒₗᵥₑₙₜ) (Table 4). Other examples for the extraction of compounds of interest from other natural sources (tomato, grape bunch and fish skin) using NADES 2 combined with the sealed system are also presented in Table 4. The resulting extracts, as well as remaining material, are depicted in FIGS. 2-5.

In an embodiment, the extract obtained from cork using NADES comprises fatty acids and oils (oleic acid, palmitic acid and stearic acid; 1-docosanol); alcohols and small acids (2-hydroxymalonic acid; 2,3-Butanediol); phenolics and aromatics (ferulic acid and derivatives, diisooctyl phthalate and analogues); terpenoids (borneol and pantolactone); sugars (D-sorbitol, D-mannonic acid) and steroids (friedelin, stigmasterol).

TABLE 4
Extracts concentration (g/mL (m_{extract}/vₛₒₗᵥₑₙₜ)) after extraction
with NADES 2 or water as solvent, using the sealed system.

| | | | Concentration of extract (g/ml) | | | |
|---|---|---|---|---|---|---|
| | Eutectic | Eutectic | | Grape | | Fish |
| Solvent | compounds | Ratio | Cork^{a)} | bunch^{b)} | Tomato^{c)} | skin^{d)} |
| H₂O | — | — | 0.0178 | 0.0455 | 0.079 | 0.1301 |
| NADES | Glyc- | 1:1 | 0.07812 | 0.0653 | 0.1161 | 0.2000 |
| 2 | erol:Lactic | | | | | |
| | acid | | | | | |

^{a)}Cork 0.7 g; Solvent 5 mL; 100° C. 6 hours; 3 cycles.
^{b)}Grape Bunch 0.7 g; Solvent 5 mL; 100° C. 6 hours; 1 cycle.
^{c)}Tomato 0.7 g; Solvent 5 mL; 100° C. 6 hours; 1 cycle.
^{d)}Fish skin 1.0 g; Solvent 5 mL; 100° C. 6 hours; 1 cycle.

When the sealed system is used as an extraction method, the extract obtained is more concentrated than the other techniques, namely the enfleurage technique and ultrasound assisted extraction (Table 4). The efficiency of the sealed system is related to the creation of pressure inside the vessel which translates into an improvement in the extraction of natural cork compounds. The enfleurage method is static, which is why the extract obtained is the least concentrated of the three extraction techniques presented in this work. The ultrasound assisted extraction have an intermediate performance in terms of extract concentration.

The present disclosure also relates to the use of a composition comprising the NADES formulations and the natural extract in cosmetic applications. Since the NADES formulations used are compatible with cosmetic application, there is no need to further purify the extract obtained.

NADES containing chemical compounds from naturals sources could be further enriched with active components for skin topical applications for sensations of warm, cold, freshness, relaxing, pain relive, lightness and well-being. In an embodiment, the composition may further comprise a topical active compound, such as icilin, menthol, carboxylated icilin, among others. These topical active compounds can induce different sensations on the skin where the composition is applied, such as listed in Table 5.

In an embodiment, the composition comprises up to 1% (w/w) of the natural deep eutectic mixture, 0.1% (w/w) of the natural extract and 97.9% (w/w) of excipients, including up to 1% (w/w) of the topical active compound.

In an embodiment, the composition can be applied in atopic skin by the inclusion of one or further components as: Hyaluronic acid, Niacinamide, Folic acid, D-Panthenol, Tocopherol, Ceramide NP (3), Ceramide AP (6 II), Ceramide EOP (1), Apigenin, Quercitin, Luteolin, Ursolic acid, Rosmarinic acid, Thymol, Carvacrol, Cooper peptide, K18 peptide, Retinol, Urea and Xylitol, or mixtures thereof.

TABLE 5
Molecular formula and chemical structure of the topical active compounds to
incorporate in NADES formulations and the respective skin sensations induced.

| | Skin | Molecular | |
|---|---|---|---|
| Compound | Feeling | formula | Chemical Structure |
| Icilin | Freshness | C₁₆H₁₃N₃O₄ | 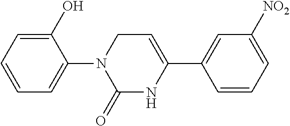 [SMILES: O=C1NC(c2cccc([N+](=O)[O-])c2)=CCN1c1ccccc1O] |
| Menthol | Freshness | C₁₀H₂₀O | 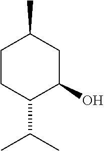 |
| Carboxylated Icilin | Freshness | C₁₇H₁₃N₃O₆ | 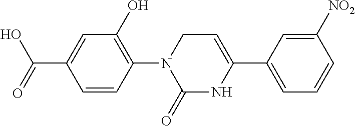 [SMILES: O=C(O)c1ccc(N2CC=C(c3cccc([N+](=O)[O-])c3)NC2=O)c(O)c1] |
| Carboxylated Menthol | Freshness | C₁₁H₂₀O₂ | 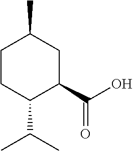 |
| 2,6- dimethylaniline | Antipain | C₈H₁₁N | 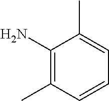 |
| Carboxyiciline- 2,6 dimethylaniline conjugate | Antipain/ Freshness | C₂₅H₂₂N₄O₅ | 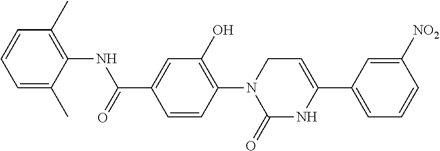 [SMILES: Cc1cccc(C)c1NC(=O)c1ccc(N2CC=C(c3cccc([N+](=O)[O-])c3)NC2=O)c(O)c1] |
| Carboxymenthol- 2,6- dimethylaniline conjugate | Antipain/ Freshness | C₁₉H₂₉NO | 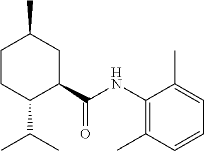 [SMILES: Cc1cccc(C)c1NC(=O)[C@@H]1C[C@H](C)CC[C@H]1C(C)C] |
| Dermorphin- derived tetrapeptide (Dmt1) DALDA | Antipain/ Relaxing | C₃₂H₄₉N₉O₅ | 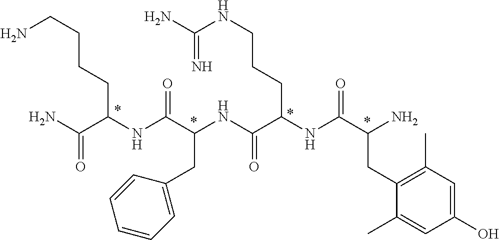 [SMILES: Cc1cc(O)cc(C)c1CC(N)C(=O)NC(CCCNC(=N)N)C(=O)NC(Cc1ccccc1)C(=O)NC(CCCCN)C(N)=O] |
| Carboxymenthol- DALDA | Antipain/ Relaxing/ Freshness | C₄₃H₆₇N₉O₆ | 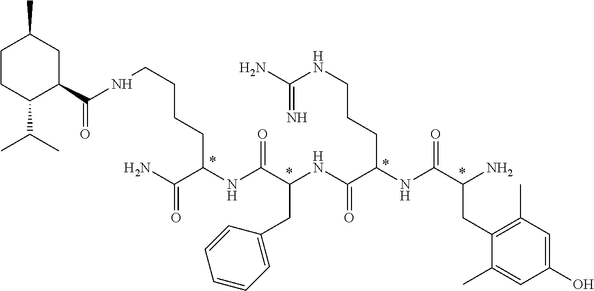 [SMILES: Cc1cc(O)cc(C)c1CC(N)C(=O)NC(CCCNC(=N)N)C(=O)NC(Cc1ccccc1)C(=O)NC(CCCCNC(=O)[C@@H]1C[C@H](C)CC[C@H]1C(C)C)C(N)=O] |
| Carboxyicilin- DALDA | Antipain/ Relaxing/ Freshness | C₄₉H₆₀N₁₂O₁₀ | 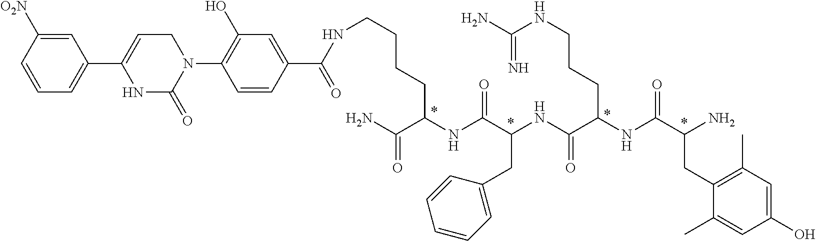 [SMILES: Cc1cc(O)cc(C)c1CC(N)C(=O)NC(CCCNC(=N)N)C(=O)NC(Cc1ccccc1)C(=O)NC(CCCCNC(=O)c1ccc(N2CC=C(c3cccc([N+](=O)[O-])c3)NC2=O)c(O)c1)C(N)=O] |
| Opioid peptides | Relaxing | YGGFL: C₂₈H₃₇N₅O₇ | 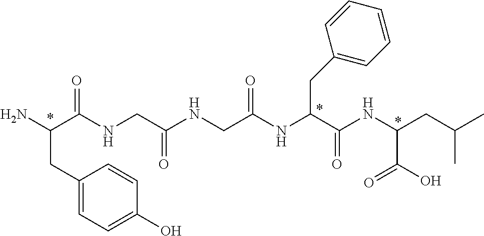 [SMILES: CC(C)CC(NC(=O)C(Cc1ccccc1)NC(=O)CNC(=O)CNC(=O)C(N)Cc1ccc(O)cc1)C(=O)O] |
| | | YPWF-NH₂: C₃₄H₃₈N₆O₅ | 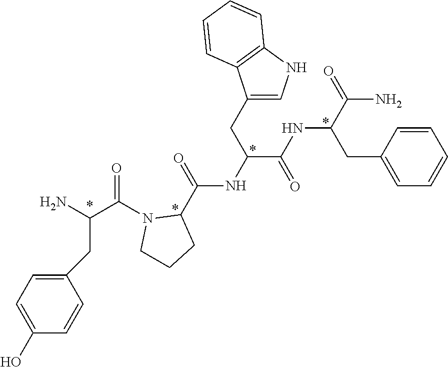 [SMILES: NC(=O)C(Cc1ccccc1)NC(=O)C(Cc1c[nH]c2ccccc12)NC(=O)C1CCCN1C(=O)C(N)Cc1ccc(O)cc1] |
| | | YGGFM: C₂₇H₃₅N₅O₇S | 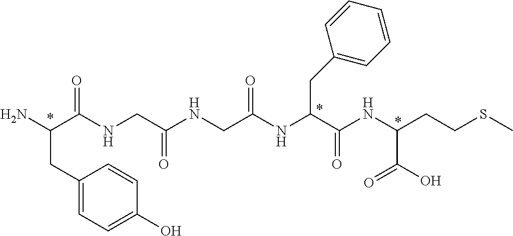 [SMILES: CSCCC(NC(=O)C(Cc1ccccc1)NC(=O)CNC(=O)CNC(=O)C(N)Cc1ccc(O)cc1)C(=O)O] |
| | | YPFF-NH₂: C₃₂H₃₇N₅O₅ | 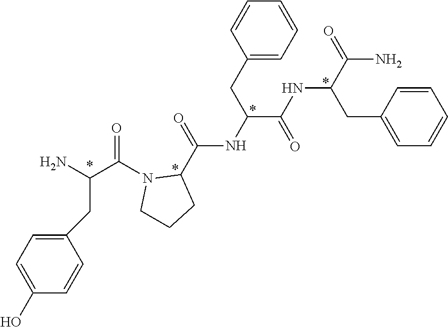 [SMILES: NC(=O)C(Cc1ccccc1)NC(=O)C(Cc1ccccc1)NC(=O)C1CCCN1C(=O)C(N)Cc1ccc(O)cc1] |
| Carboxymenthol- YGGFL conjugate | Freshness/ Relaxing | C₃₉H₅₅N₅O₈ | 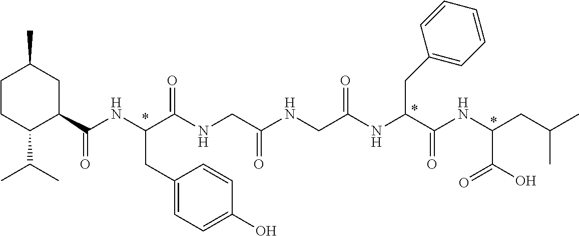 [SMILES: CC(C)CC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O] |
| Carboxymenthol- YGGFM conjugate | Freshness/ Relaxing | C₃₈H₅₃N₅O₈S | 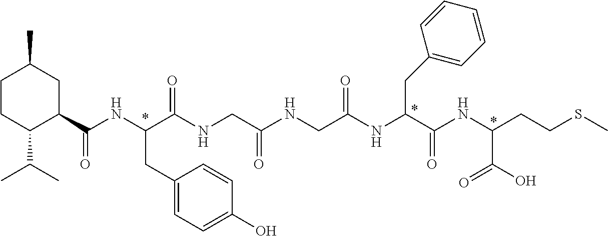 [SMILES: CSCCC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O] |
| Carboxymenthol- YPWF-NH2 conjugate | Freshness/ Relaxing | C₄₅H₅₆N₆O₆ | 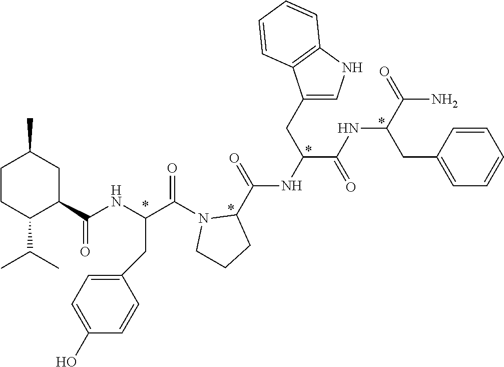 [SMILES: CC(C)[C@@H]1CC[C@@H](C)C[C@H]1C(=O)NC(Cc1ccc(O)cc1)C(=O)N1CCCC1C(=O)NC(Cc1c[nH]c2ccccc12)C(=O)NC(Cc1ccccc1)C(N)=O] |
| Carboxymenthol- YPFF-NH₂ conjugate | Freshness/ Relaxing | C₄₃H₅₅N₅O₆ | 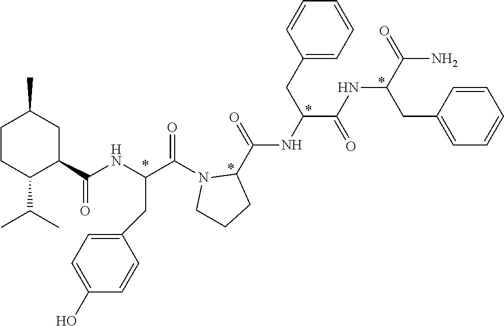 [SMILES: CC(C)[C@@H]1CC[C@@H](C)C[C@H]1C(=O)NC(Cc1ccc(O)cc1)C(=O)N1CCCC1C(=O)NC(Cc1ccccc1)C(=O)NC(Cc1ccccc1)C(N)=O] |
| Carboxyicilin- YGGFL conjugate | Freshness/ Relaxing | C₄₅H₄₈N₈O₁₂ | 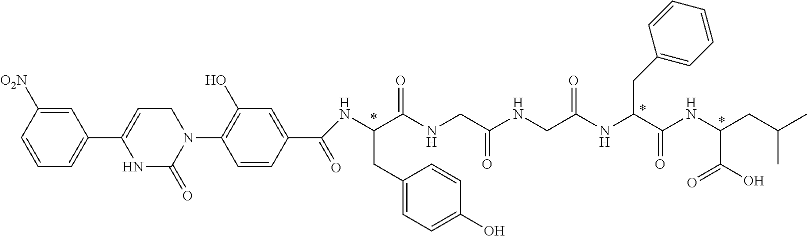 [SMILES: CC(C)CC(NC(=O)C(Cc1ccccc1)NC(=O)CNC(=O)CNC(=O)C(Cc1ccc(O)cc1)NC(=O)c1ccc(N2CC=C(c3cccc([N+](=O)[O-])c3)NC2=O)c(O)c1)C(=O)O] |
| Carboxyicilin- YGGFM conjugate | Freshness/ Relaxing | C₄₄H₄₆N₈O₁₂S | 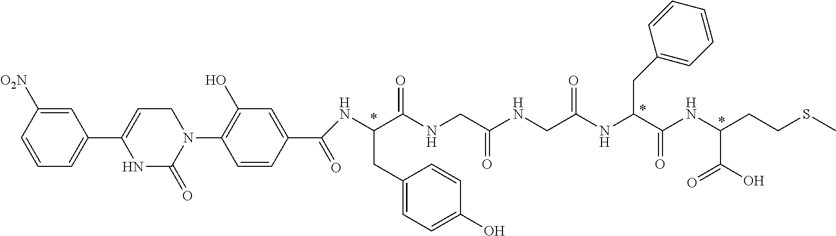 [SMILES: CSCCC(NC(=O)C(Cc1ccccc1)NC(=O)CNC(=O)CNC(=O)C(Cc1ccc(O)cc1)NC(=O)c1ccc(N2CC=C(c3cccc([N+](=O)[O-])c3)NC2=O)c(O)c1)C(=O)O] |
| Carboxyicilin- YPWF-NH₂ conjugate | Freshness/ Relaxing | C₅₁H₄₉N₉O₁₀ | 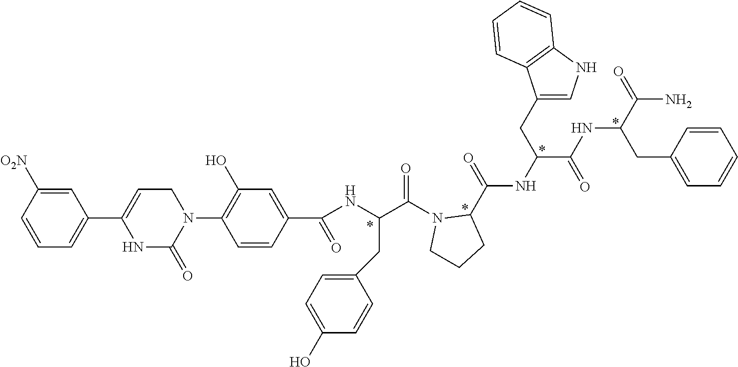 [SMILES: NC(=O)C(Cc1ccccc1)NC(=O)C(Cc1c[nH]c2ccccc12)NC(=O)C1CCCN1C(=O)C(Cc1ccc(O)cc1)NC(=O)c1ccc(N2CC=C(c3cccc([N+](=O)[O-])c3)NC2=O)c(O)c1] |
| Carbocyicilin- YPFF- NH₂ conjugate | Freshness/ Relaxing | C₄₉H₄₈N₈O₁₀ | 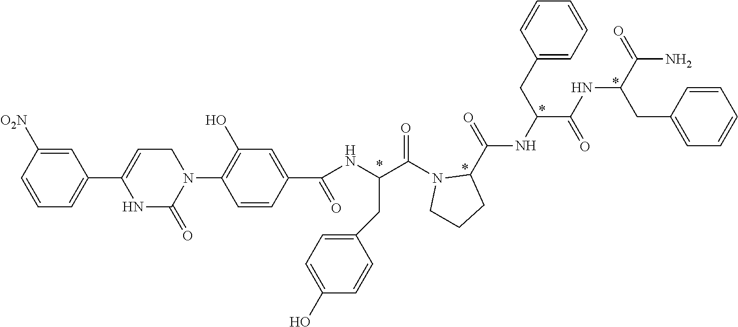 [SMILES: NC(=O)C(Cc1ccccc1)NC(=O)C(Cc1ccccc1)NC(=O)C1CCCN1C(=O)C(Cc1ccc(O)cc1)NC(=O)c1ccc(N2CC=C(c3cccc([N+](=O)[O-])c3)NC2=O)c(O)c1] |
| Sivelestat | Anti- wrinkle/ Anti-aging | C₂₀H₂₂N₂O₇S | 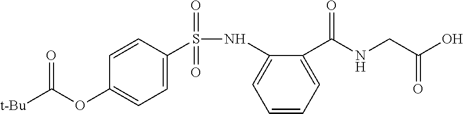 [SMILES: CC(C)(C)C(=O)Oc1ccc(S(=O)(=O)Nc2ccccc2C(=O)NCC(=O)O)cc1] |
| Argireline Ac- EEMQRR- NH2 | Anti- wrinkle/ Anti-aging | C₄₆H₅₆N₁₂O₆ | 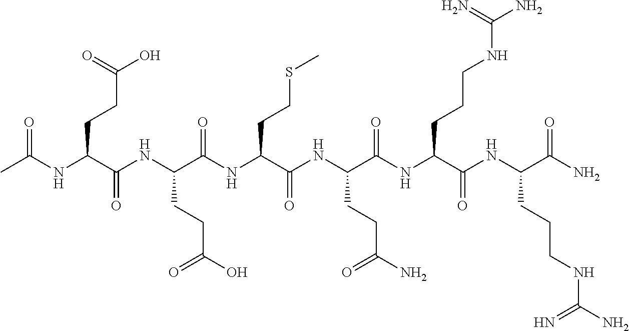 [SMILES: CSCC[C@H](NC(=O)[C@H](CCC(=O)O)NC(=O)[C@H](CCC(=O)O)NC(C)=O)C(=O)N[C@@H](CCC(N)=O)C(=O)N[C@@H](CCCNC(=N)N)C(=O)N[C@@H](CCCNC(=N)N)C(N)=O] |
| Sivelestate- argireline Ac- EEMQRR- NH2 | Anti- wrinkle/ Anti-aging | C₅₂H₇₈N₁₆O₁₇S₂ | 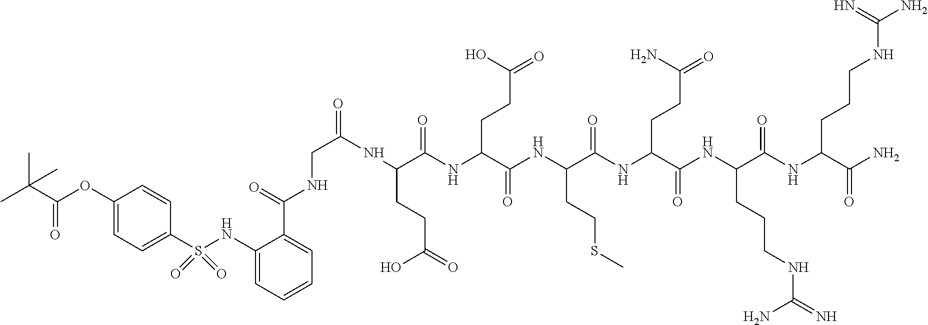 [SMILES: CSCCC(NC(=O)C(CCC(=O)O)NC(=O)C(CCC(=O)O)NC(=O)CNC(=O)c1ccccc1NS(=O)(=O)c1ccc(OC(=O)C(C)(C)C)cc1)C(=O)NC(CCC(N)=O)C(=O)NC(CCCNC(=N)N)C(=O)NC(CCCNC(=N)N)C(N)=O] |

Sequence List:
SEQ ID No 1: Tyr-Gly-Gly-Phe-Leu (YGGFL);
SED ID No 2: Tyr-Pro-Trp-Phe (YPWF);
SEQ ID No 3: Tyr-Gly-Gly-Phe-Met (YGGFM);
SEQ ID No 4: Tyr-Pro-Phe-Phe (YPFF);
SEQ ID No 5: Glu-Glu-Met-Gln-Arg-Arg (EEMQRR);
SEQ ID No 6: Xaa Arg Phe Lys (XRFK) wherein Xaa=Tyr(2,6-dimethyl)

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following claims further set out particular embodiments of the disclosure.

### REFERENCES

[1] Choi, Y. H., Spronsen, J., Dai, Y., Verberne, M., Hollmann, F., Arends, I. W. C. E., Witkamp, G., Verpoorte, R., Plant Physiology 2011, 156, 1701-1705.
[2] Dai, Y., Spronsen, J., Witkamp, G., Verpoorte, R., Choi, Y. H., Journal of Natural Products 2013, 76, 2162-2173.
[3] Dai, Y., Spronsen, J., Witkamp, G., Verpoorte, R., Choi, Y. H., Analytica Chimica Acta 2013, 766, 61-68.
[4] Vanda, H., Dai, Y., Wilson, E. G., Verpoorte, R., Choi, Y. H., Comptes Rendus Chimie 2018, 21, 628-638.
[5] Zdanowicz, M., Wilpiszewska, K., Spychaj, T., Carbohydrate Polymers 2018, 200, 361-380.
[6] Zhang, Q., Vigier, K. O., Royer, S., Jerome, F., Chemical Society Reviews 2012, 41, 7108-7146.
[7] Tang, B., Zhang, H., Row, K. H., Journal of Separation Science 2015, 38, 1053-1064.
[8] Zahrima, I., Nasikin, M., Krisanti, E., Mulia, K., Food Chemistry 2018, 240, 490-496.
[9] Dai, Y., Verpoorte, R., Choi, Y. H., Food Chemistry 2014, 159, 116-121.
[10] Dai, Y., Witkamp, G., Verpoorte, R., Choi, Y. H., Analytical Chemistry 2013, 85, 6272-6278.
[11] Kalhor, P., Ghandi, K., Molecules 2019, 200, 159-166.
[12] Bubalo, M. C., Curko, N., Tomasevic, M., Ganic, K. K., Redovnikovic, I. R., Food Chemistry 2016, 159, 116-121.
[13] Ruesgas-Ramon, M., Figueroa-Espinoza, M. C., Durand, E., Journal of Agricultural Food Chemistry 2017, 65, 3591-3601.
[14] Cunha, S. C., Fernandes, J. O., Trends in Analytical Chemistry 2018, 105, 225-239.
[15] Yunjian, Ma, Peilin Li, Yongru Li, Willot, S. J. P., Zhang W., Ribitsch, D., Choi Y. H., Verpoorte R., Zhang T., Hollmann F., Wang Y., ChemsusChem 2019, 12, 1310-1315.
[16] Laguerre, M.; Lavaud, A., Naturex 2016, 45-47.
[17] Jin, Y., Jung, D., Li, K., Park, K., Ko, J., Y, M., Lee, J., Applied Sciences 2019, 9, 2581-2590.
[18] Wiles, R., Crow, G., Pain, H., Qualitative Research 2011, 11, 587-604.
[19] Khezeli, T., Daneshfar, A., Sahraei, R., Journal of Chromatography A 2015, 1425, 25-33.
[20] Pereira, H., Wood Science Technology 1988, 22, 211-218.
[21] Salomé-Ararca, L. F., Soto-Hernandez, R. M., Cruz-Huerta, N., Gonzalez-Hernandez, V. A., Botanical sciences 2015, 93 (3), 633-638.

1. Natural deep eutectic mixture for extraction of biocomponents comprising two different solvents, wherein
a first solvent is selected from a list consisting of: lactic acid, and glycerol, citric acid, maleic acid, and tetrabutylammonium bromide;
and a second solvent is selected from a list consisting of: sodium lactate, sodium citrate, transcutol, glycine, glycerol, oleic acid, sodium lactate, decanoic acid;
further comprising up to 90% (w/w) in water.

2. Natural deep eutectic mixture according to claim 1 comprising:
20-90% (mol/mol) of the first solvent; preferably 30-80% (mol/mol);
10-80% (mol/mol) of the second solvent, preferably 12-50% (mol/mol).

3. Natural deep eutectic mixture according to claim 1 wherein the natural deep eutectic mixture is selected from the following list: lactic acid and glycerol; lactic acid and sodium citrate; glycerol and sodium lactate; glycerol and transcutol; lactic acid and transcutol; transcutol and sodium lactate; lactic acid and glycine; tetrabutylammonium bromide and oleic acid; tetrabutylammonium bromide and decanoic acid; preferably the natural deep eutectic mixture is a combination of: glycerol and transcutol, or lactic acid and transcutol, glycerol and maleic acid.

4. Natural deep eutectic mixture according to claim 1 comprising the equivalent molar ratio between the first and the second solvent ranges from 1:4 to 4:1, preferably (1:4 to 1:3); more preferably 1:1.

5. Natural deep eutectic mixture according to claim 1 comprising lactic acid and glycerol in an equivalent molar ratio of 1:4 to 4:1; preferably (1:4-1:3).

6. Natural deep eutectic mixture according to claim 1 comprising 4:1; lactic acid and sodium citrate in an equivalent molar ratio of 2:1 to 8:1.

7. Natural deep eutectic mixture according to claim 1 comprising 2:1; glycerol and sodium lactate, in an equivalent molar ratio of 1:2 to 2:1.

8. Natural deep eutectic mixture according to claim 1 comprising 1:2; citric acid and sodium lactate in an equivalent molar ratio of 1:4 to 1:3.

9. Natural deep eutectic mixture according to claim 1 comprising lactic acid and glycine in an equivalent molar ratio of 5:1.

10. Natural deep eutectic mixture according to claim 1 comprising tetrabutylammonium bromide and oleic acid, or tetrabutylammonium bromide and decanoic acid, or maleic acid and ethylene glycol, equivalent molar ratio of 1:2.

11. Natural deep eutectic mixture according to claim 1 comprising 6 to 20% (w/w) in water.

12. Natural deep eutectic mixture according to claim 1 wherein the mixture is clear and liquid at a temperature ranging from 15 to 30° C. and the melting point ranges from −55 to −15° C., preferably from −52 to −20° C.

13. Natural deep eutectic mixture according to claim 1 wherein the is adjustable by changing the molar ratio between the two different solvents and/or by the addition of sodium hydroxide, preferably 10 to 30% (w/w) (weight of sodium hydroxide/weight of mixture).

14. Natural deep eutectic mixture according to claim 1 wherein the density of the mixture ranges from 1.2 to 1.4 g·ml⁻¹, and/or the viscosity ranges from 0.015 to 1700 Pa·s at 25° C.

15. Method to obtain an extract from a natural source material, comprising the following steps:
contacting, preferably dipping, the natural source material with a natural deep eutectic mixture described in claim 1;
incubating the natural deep eutectic mixture and the natural source material at a temperature ranging from 25° C. to 150° C. during 1 min to 24 hours, preferably 25 to 100° C.;
replacing the used natural source material by a new natural source material; repeating the previous steps for 0 to 30 times;
optionally wherein the natural source material is selected from a list comprising: cork, agricultural wastes, tomato, olive oil, grape seeds, grape peels, plants, teas, eucalyptus, lavender, fish skin or bones, or mixtures thereof.

16. Method according to claim 15 wherein the natural source material is cork.

17. Method according to claim 15 further comprising a step of increasing the viscosity of the natural deep eutectic mixture by reaction with a lipase, esterase or protease.

18. Extract obtainable by the method described in claim 15 comprising fatty acids and oils, preferably oleic acid, palmitic acid, stearic acid, 1-docosanol; alcohols and small acids, such as 2-hydroxymalonic acid, 2,3-Butanediol; phenolics and aromatics, such as ferulic acid and derivatives, diisooctyl phthalate or analogues; terpenoids, such as borneol or pantolactone; sugars, such as D-sorbitol, D-mannonic acid; steroids, such as friedelin, stigmasterol, or mixtures thereof.

19. Composition comprising a natural deep eutectic mixture as described in claim 1 and at least one of the following: an extract comprising fatty acids and oils, preferably oleic acid, palmitic acid, stearic, acid, 1-docosanol, alcohols and small acids, such as 2-hydroxymalonic acid, 2,3-Butanediol, phenolics and aromatics, such as ferulic acid and derivatives, diisooctyl phthalate or analogues: terpenoids, such as borneol or pantolactone, sugars, such as D-sorbitol, D-mannonic acid; steroids, such as friedelin, stigmasterol: or mixtures thereof, or up to 1% (w/w) of topical active compound.

20. Composition according to claim 19 comprising 0.01-1% (w/w) of the topical active compound, preferably 0.1-0.5 (w/w) of the topical active compound.

21. Composition according to claim 20 comprising up to 90% (w/w) of the natural deep eutectic mixture, up to 10% (w/w) of the extract and up to 1% (w/w) of the topical active compound.

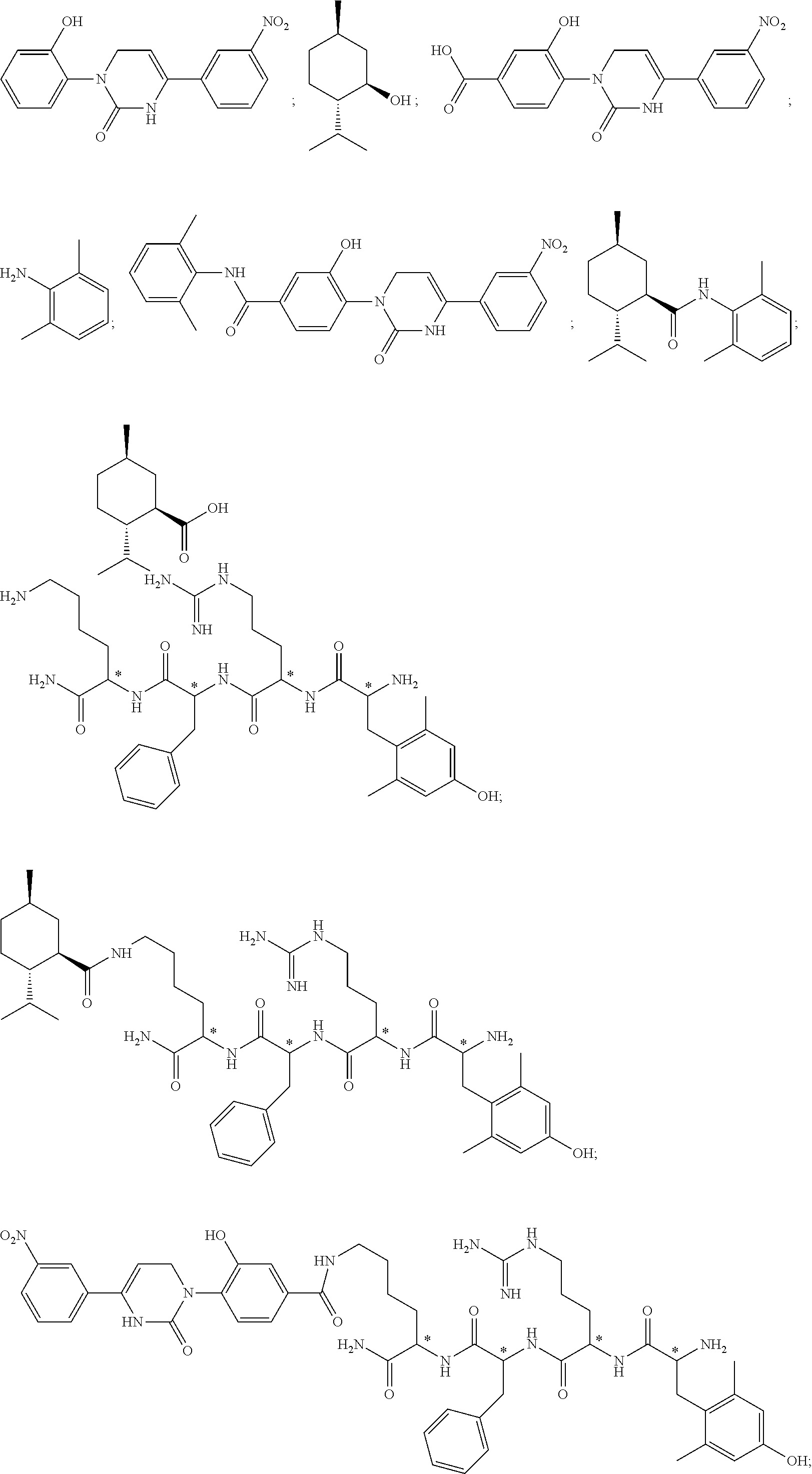

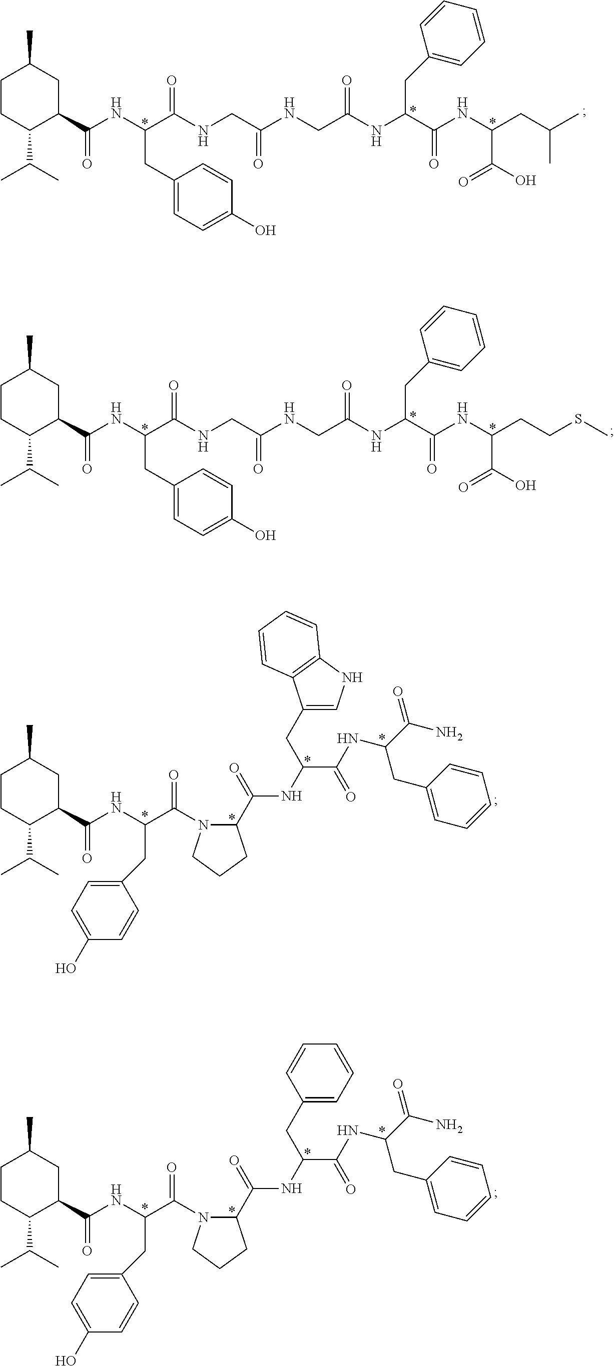 [SMILES: CC(C)CC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O ; CSCCC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O ; CC(C)[C@@H]1CC[C@@H](C)C[C@H]1C(=O)NC(Cc1ccc(O)cc1)C(=O)N1CCCC1C(=O)NC(Cc1c[nH]c2ccccc12)C(=O)NC(Cc1ccccc1)C(N)=O ; CC(C)[C@@H]1CC[C@@H](C)C[C@H]1C(=O)NC(Cc1ccc(O)cc1)C(=O)N1CCCC1C(=O)NC(Cc1ccccc1)C(=O)NC(Cc1ccccc1)C(N)=O]

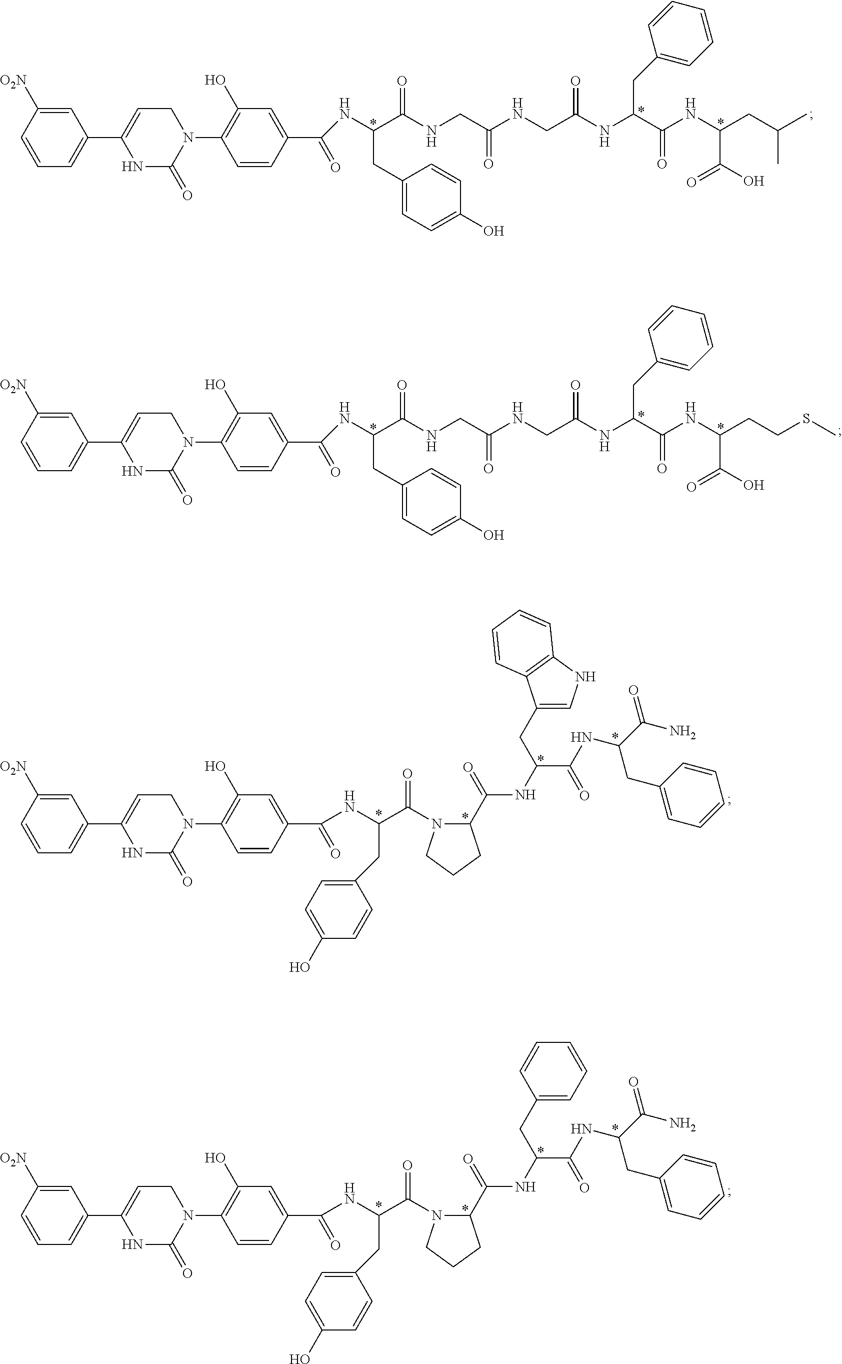

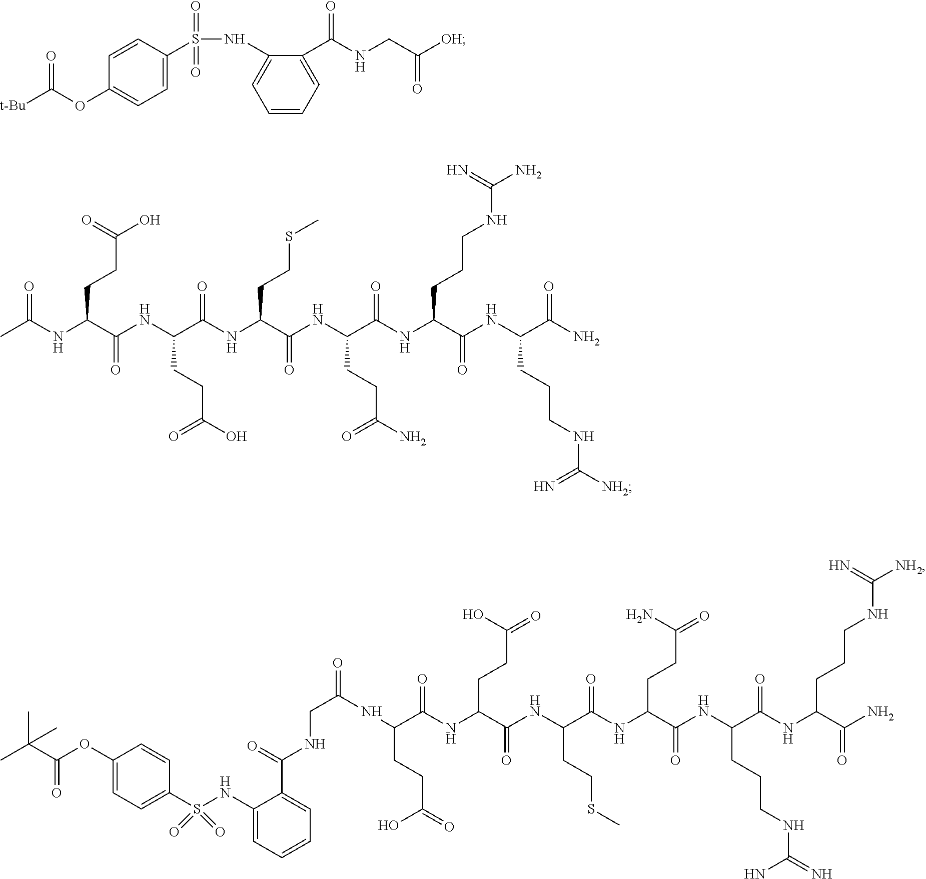 [SMILES: CC(C)(C)C(=O)Oc1ccc(S(=O)(=O)Nc2ccccc2C(=O)NCC(=O)O)cc1 ; CSCC[C@H](NC(=O)[C@H](CCC(=O)O)NC(=O)[C@H](CCC(=O)O)NC(C)=O)C(=O)N[C@@H](CCC(N)=O)C(=O)N[C@@H](CCCNC(=N)N)C(=O)N[C@@H](CCCNC(=N)N)C(N)=O ; CSCCC(NC(=O)C(CCC(=O)O)NC(=O)C(CCC(=O)O)NC(=O)CNC(=O)c1ccccc1NS(=O)(=O)c1ccc(OC(=O)C(C)(C)C)cc1)C(=O)NC(CCC(N)=O)C(=O)NC(CCCNC(=N)N)C(=O)NC(CCCNC(=N)N)C(N)=O]

22. Composition according to claim 19 further comprising a component selected from a list consisting of: hyaluronic acid, niacinamide, folic acid, D-panthenol, tocopherol, ceramide NP (3), ceramide AP (6 II), ceramide FOP (1), apigenin, quercitin, luteolin, ursolic acid, rosmarinic acid, thymol, carvacrol, cooper peptide, K18 peptide, retinol, urea, xylitol, or mixtures thereof.

23. Use of the composition as described in claim 19 as a cosmetic formulation or in hair treatment, namely hair products, preferably in hair conditioners, hair curling agents, hair straightening agents, hair masks, or hair shampoos; or in skin care treatment, namely skin care products, preferably in skin balms, skin creams, skin soap, skin masks, or skin moisturizers.

24. Composition according to claim 19 wherein the topical active compound is selected from a list comprising: opioid peptide, secretory leukocyte protease inhibitor; 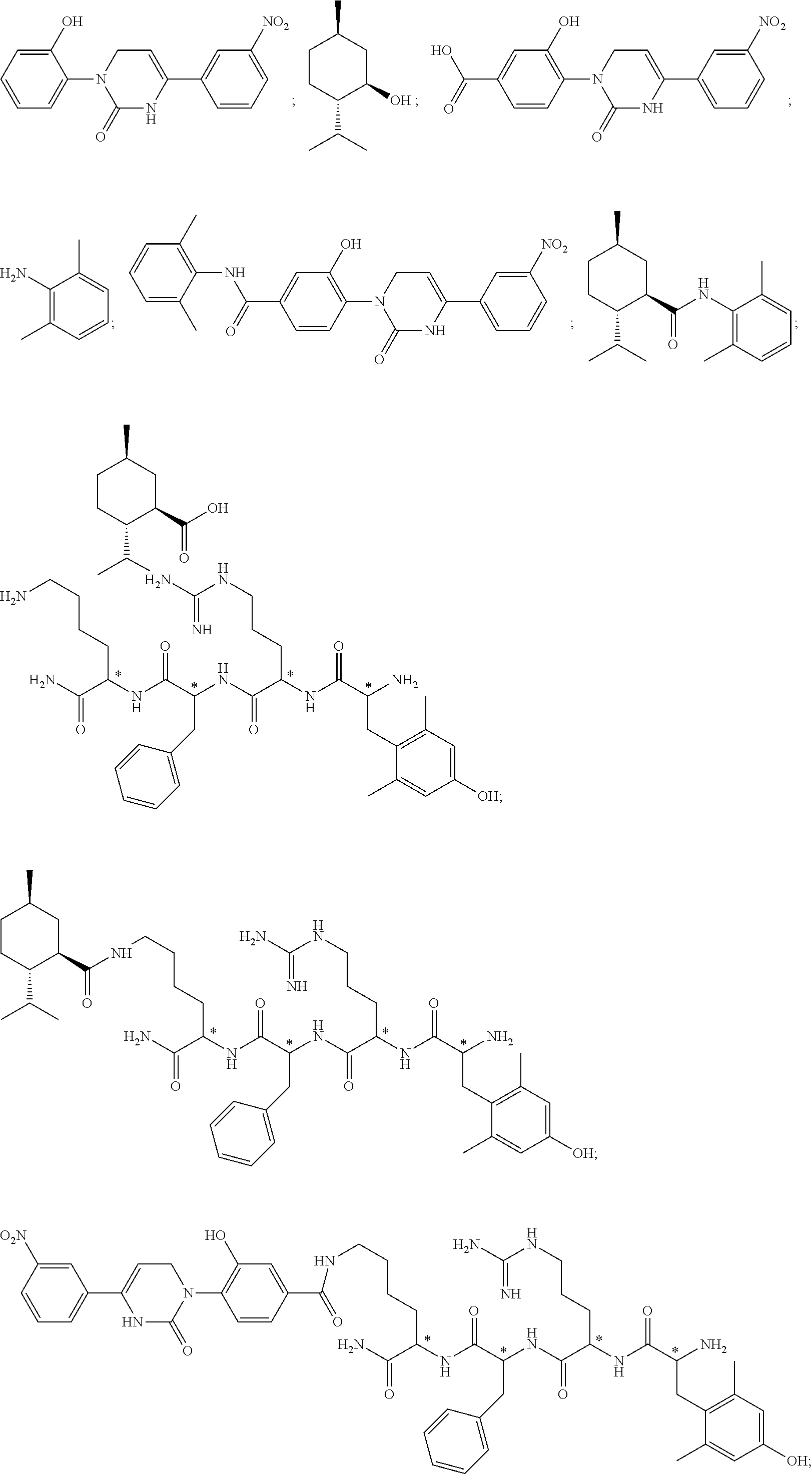 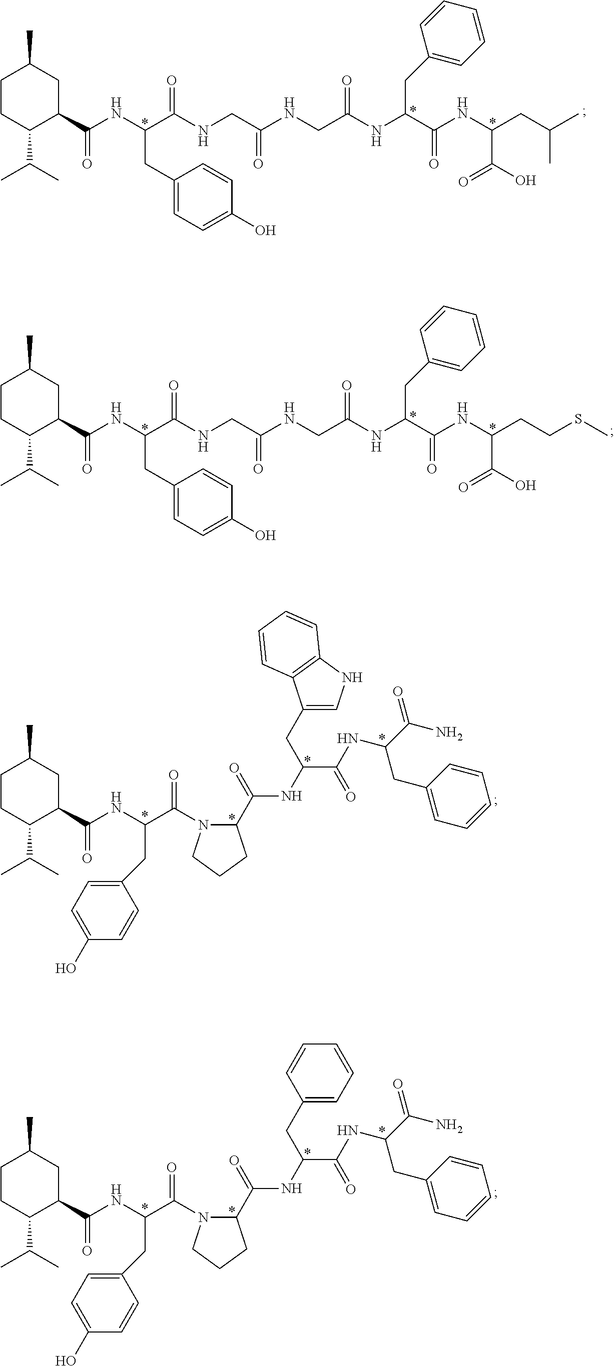 [SMILES: CC(C)CC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O ; CSCCC(NC(=O)C(Cc1ccccc1)NC(=O)CNC(=O)CNC(=O)C(Cc1ccc(O)cc1)NC(=O)[C@@H]1C[C@H](C)CC[C@H]1C(C)C)C(=O)O ; CC(C)[C@@H]1CC[C@@H](C)C[C@H]1C(=O)NC(Cc1ccc(O)cc1)C(=O)N1CCCC1C(=O)NC(Cc1c[nH]c2ccccc12)C(=O)NC(Cc1ccccc1)C(N)=O ; CC(C)[C@@H]1CC[C@@H](C)C[C@H]1C(=O)NC(Cc1ccc(O)cc1)C(=O)N1CCCC1C(=O)NC(Cc1ccccc1)C(=O)NC(Cc1ccccc1)C(N)=O] 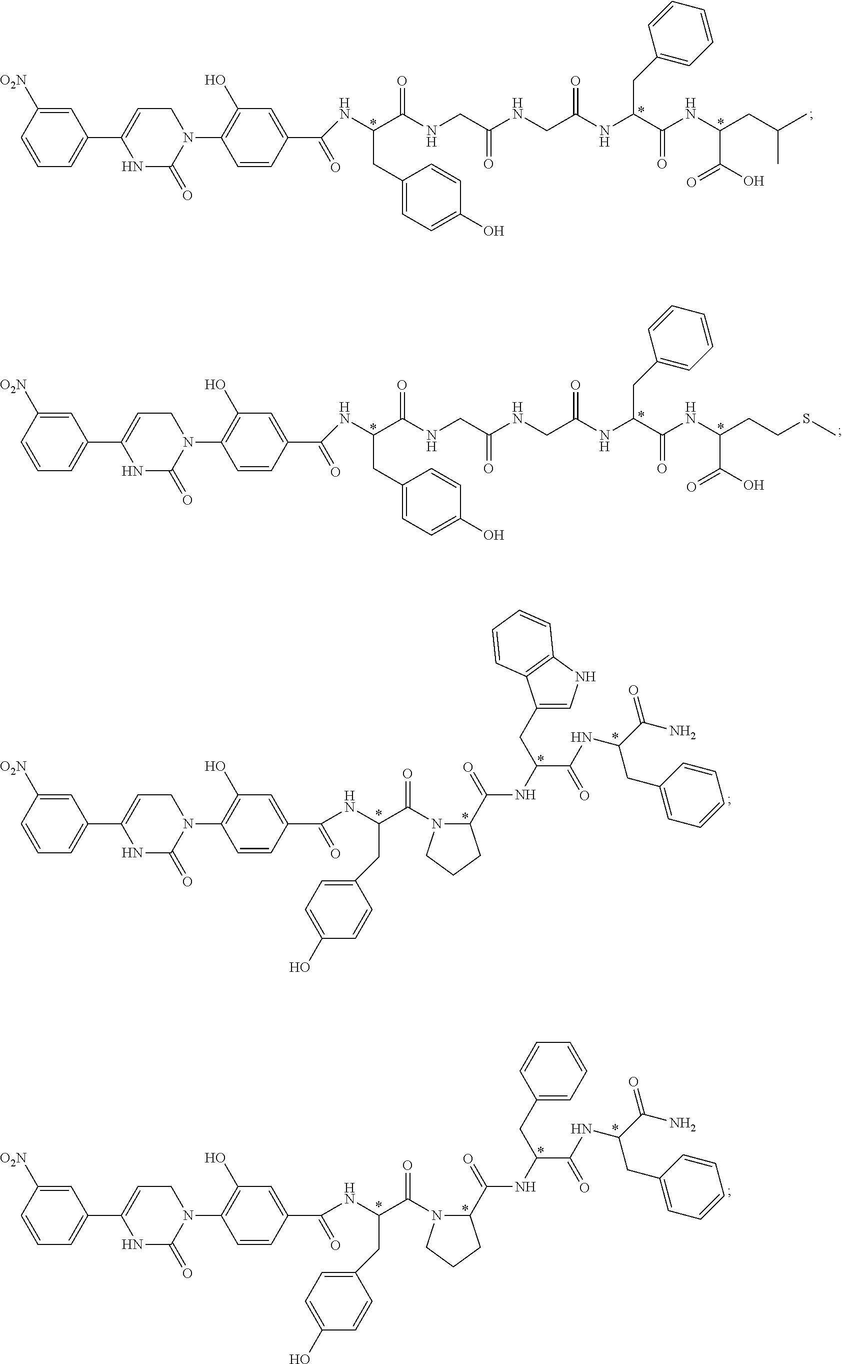 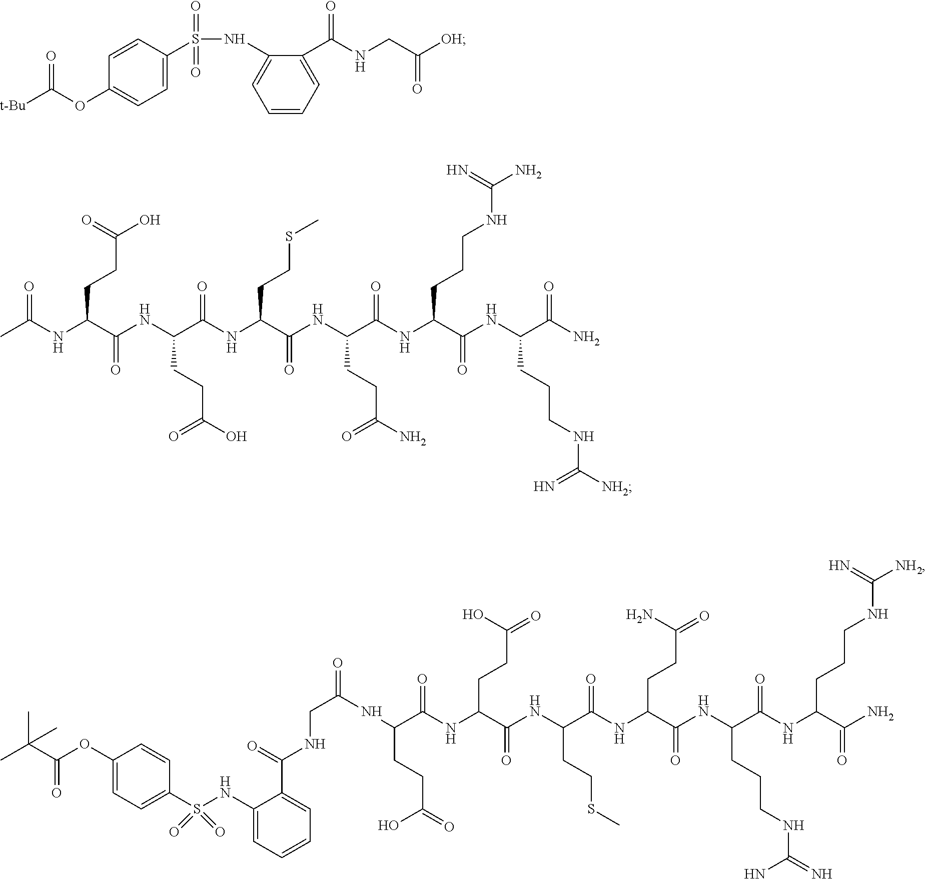 [SMILES: CC(C)(C)C(=O)Oc1ccc(S(=O)(=O)Nc2ccccc2C(=O)NCC(=O)O)cc1 ; CSCC[C@H](NC(=O)[C@H](CCC(=O)O)NC(=O)[C@H](CCC(=O)O)NC(C)=O)C(=O)N[C@@H](CCC(N)=O)C(=O)N[C@@H](CCCNC(=N)N)C(=O)N[C@@H](CCCNC(=N)N)C(N)=O ; CSCCC(NC(=O)C(CCC(=O)O)NC(=O)C(CCC(=O)O)NC(=O)CNC(=O)c1ccccc1NS(=O)(=O)c1ccc(OC(=O)C(C)(C)C)cc1)C(=O)NC(CCC(N)=O)C(=O)NC(CCCNC(=N)N)C(=O)NC(CCCNC(=N)N)C(N)=O]
or mixtures thereof.


SEQUENCE LISTING (6 sequences)

SEQ ID NO: 1 - protein, 5 aa, Artificial Sequence
  opioid peptide
YGGFL

SEQ ID NO: 2 - protein, 4 aa, Artificial Sequence
  opioid peptide
YPWF

SEQ ID NO: 3 - protein, 5 aa, Artificial Sequence
  opioid peptide
YGGFM

SEQ ID NO: 4 - protein, 4 aa, Artificial Sequence
  opioid peptide
YPFF

SEQ ID NO: 5 - protein, 6 aa, Artificial Sequence
  EEMQRR
EEMQRR

SEQ ID NO: 6 - protein, 4 aa, Artificial Sequence
  MOD_RES 1: METHYLATION Tyr(2,6-dimethyl)
  (Dmt1) DALDA
XRFK